(19) United States
(12) Patent Application Publication
(10) Pub. No.: US 2026/0295286 A1
(43) Pub. Date: Oct. 1, 2026

(54) Modular Light Therapy Panel and An Apparatus for Mounting the Light Therapy Panel

(71) Applicant: Shenzhen Rainbow Light Medical Equipment Co., Ltd, Shenzhen (CN)

(72) Inventors: ALAIN DIJKSTRA, Amstelveen (NL); YIN ZHICHAO, Shenzhen (CN); CAO JIAMING, Shenzhen (CN); JORDAN VAN HEMERT, Melbourne (AU); Huadong Yuan, Shenzhen (CN); Zun Qiu, Shenzhen (CN)

(73) Assignee: Shenzhen Rainbow Light Medical Equipment Co., Ltd, Shenzhen (CN)

(21) Appl. No.: 19/409,811

(22) Filed: Dec. 5, 2025

(30) Foreign Application Priority Data
Mar. 25, 2025 (CN) 2025205312111
Jun. 4, 2025 (CN) 2025211322233

(51) Int. Cl. A61N 5/06; A61N 5/00

(52) U.S. Cl. CPC A61N 5/06; A61N 2005/005; A61N 2005/0633; A61N 2005/0652

(57) ABSTRACT

Embodiments of the present invention provide a modular light therapy panel and system that enables the interconnection of multiple panels using a reinforced bracket secured by a plurality of knobs. The modular light therapy panel comprises an interlocking mounting slot that extends horizontally and has a curved, wave-like contour with multiple circular cutouts, each containing a mounting hole for securing the panel with a threaded knob. A bracket is positioned over one of the circular cutouts to connect two adjacent panels. The bracket is secured by tightening a first knob into the mounting hole of the first panel and a second knob into the mounting hole of the second panel, ensuring a stable and expandable configuration. The modular light therapy is further designed for attachment to a stand assembly.

### TECHNICAL FIELD

The present invention relates generally to modular light therapy panels designed for therapeutic applications. More specifically, the present invention provides a system that enables seamless connectivity between multiple light panels. The system is equipped with multiple mounting options, enhanced mobility features, and efficient heat dissipation mechanisms, making it suitable for both clinical and personal use.

### BACKGROUND

Light therapy is widely recognized for its therapeutic benefits in managing various health conditions, including seasonal affective disorder (SAD), sleep disturbances, circadian rhythm irregularities, skin conditions, and mood enhancement. Exposure to specific wavelengths of light has been shown to influence melatonin levels, regulate the body's internal clock, and contribute to overall well-being. Despite the growing adoption of light therapy, existing solutions often lack flexibility in terms of mobility, portability, and adaptability to different user environments.

Conventional light therapy devices typically feature standalone panels or fixed lightboxes, requiring users to remain in a specific position for effective exposure. These units are often bulky, difficult to adjust, and cannot be repositioned or expanded according to individual preferences. While some advanced models offer limited tilting or mounting options, they do not incorporate a truly modular design that allows users to customize their setup based on available space or therapeutic needs.

While various light therapy products are available commercially, they frequently lack essential features such as seamless modular connectivity, adjustable positioning, and effortless mobility. Existing designs do not provide an effective means to interconnect multiple panels for enhanced coverage, nor do they offer convenient relocation options within a home, office, or clinical setting. Furthermore, the absence of portable features in most models makes it challenging for users to move or reconfigure their light therapy system as needed.

Current solutions are often limited to single-panel designs with restricted placement options or involve complex installations that are neither user-friendly nor convenient. There is no comprehensive system available that facilitates the connection of multiple panels in both horizontal and vertical orientations while maintaining ease of use and adaptability. The conventional method of connecting two panels is by tightening screws and connecting through a bracket. However, the connection between the panels is loose and not stable.

In order to overcome the limitations, the present invention provides a modular light therapy panel system with multiple mounting options, expandable connectivity, and enhanced mobility. The design enables users to link multiple panels using brackets, fasteners, and rotating fixtures in both horizontal and vertical configurations. The modular light therapy panel is provided with an interlocking mounting slot on the back surface, spanning horizontally and having a curved, wave-like contour with two or more circular cutouts. For connecting the two panels, the bracket is placed over the interlocking mounting slot on each panel and tightened by a screwed knob, where the circular cutout is placed with a shaft. The placement of the shaft in the circular cutout ensures that the connection between the two light therapy panels is stable and the panel does not wobble. The system provides a stand capable of supporting one or more panels, ensuring both stability and portability. Furthermore, a hanging mechanism enables users to mount the panels on doors or walls, offering a space-saving installation alternative.

### OBJECTS OF THE INVENTION

Some of the objects of the invention are as follows:

An object of the present invention is to provide a modular light therapy panel system that provides enhanced adaptability, ease of installation, and mobility.

Another object of the present invention is to provide a light therapy system that allows multiple panels to be interconnected in both horizontal and vertical orientations, enabling users to expand the treatment area as needed.

Another object of the invention is to provide a modular light therapy panel that provides flexible mounting configurations, including wall mounting, door hanging, and attachment to a stand, ensuring versatility.

Another object of the present invention is to provide a movable stand for mounting the modular light therapy panel.

Another object of the present invention is to provide a modular light therapy panel integrated with a hanging mechanism that allows panels to be mounted on doors or walls.

### SUMMARY OF THE INVENTION

According to a first aspect of the present invention, a modular light therapy panel is provided. The modular light therapy panel comprising: a housing having a front surface and a back surface; a light source disposed within the housing; a control assembly configured to regulate operation of the light source; at least one interlocking mounting slot formed on the back surface, the interlocking mounting slot having a curved, wave-shaped contour with a plurality of circular cutouts, each of the plurality of circular cutout defining one or more screw hole; wherein the interlocking mounting slot is configured to receive one or more mounting members for attachment of the modular light therapy panel to a connecting bracket or a mounting bracket.

In one embodiment of the invention, the mounting member comprise a knob and a mounting base with a through-hole; and the connecting bracket or the mounting bracket is secured over the interlocking mounting slot by placing the mounting base in the circular cut-out and allowing a threaded shaft of the knob to pass through a through hole in the connecting bracket/mounting bracket and the mounting base and tightened in the one or more screw holes.

In one embodiment of the invention, the mounting bracket comprises one or more bracket bodies comprising: a connector having a first end and a second end; a mounting plate connected to the first end and the configured to engage the interlocking mounting slot through the one or more mounting member; a hook connected to the second end and configured to attach to a supporting structure

In one embodiment of the invention, the connector includes a length adjustment member configured to adjust length between the mounting plate and the hook.

In one embodiment of the invention, the interlocking mounting slots facilitate carrying of the modular therapy panel by a user, wherein the user can insert fingers for holding the modular light therapy panel.

In one embodiment of the invention, the connecting bracket is positioned over one of the plurality of circular cut-outs of the modular light therapy panel; and an adjacent modular light therapy panel; and the connecting bracket is configured to connect the modular light therapy panel and the adjacent modular light therapy panel using the one or more mounting members.

In one embodiment of the invention, the modular light therapy panel further comprising a plurality of pre-drilled holes on the back surface for connecting a modular connecting board; and wherein the modular connecting board includes one or more connecting hooks configured to engage with a predefined slot in a stand bracket of a stand assembly.

According to a second aspect of the present invention, a modular lighting panel system is provided. The modular lighting panel system comprising: a plurality of modular light therapy panels, each panel including: a housing containing a light source on a front surface and a control assembly for controlling the light source; a back surface configured for attachment to an adjacent modular light therapy panel, the back surface comprising: at least one interlocking mounting slot positioned at the back surface, extending horizontally across the back surface; the interlocking mounting slot having a curved, wave-like contour with a plurality of circular cutouts, each cutout including a screw hole for receiving a threaded shaft of a mounting member; and a connecting bracket positioned over one of the plurality of circular cut-outs of a first modular light therapy panel and a second modular light therapy panel, wherein the connecting bracket is secured by tightening a first mounting member into a first screw hole of the first modular light therapy panel and a second mounting member into a second mounting hole of the second modular light therapy panel.

In one embodiment of the invention, the plurality of circular cutouts includes integrated metal bushings or threaded inserts to enhance durability and resistance to wear during repeated attachment and detachment cycles.

In one embodiment of the invention, the connecting bracket features elongated slots to allow for positional adjustment between the first modular light therapy panel and the second modular light therapy panel.

In one embodiment of the invention, the mounting member includes a spring-loaded locking mechanism to prevent unintended loosening due to vibration or movement during operation.

In one embodiment of the invention, the plurality of modular light therapy panels are configured to allow horizontal and vertical expansion enables customizable therapy configurations.

According to a third aspect of the present invention, a modular light therapy panel system is provided. The modular light therapy panel system comprises: a housing having a light source on a front surface and a control assembly to control the light source; a back surface configured to attach with a stand assembly or a second light therapy panel, wherein the back surface comprises: at least one interlocking mounting slot on the back surface; a plurality of pre-drilled holes for connecting a modular connecting board; and wherein the modular connecting board includes one or more connecting hooks configured to engage with a predefined slot in a stand bracket of a stand assembly.

In one embodiment of the invention, the modular connecting board is secured to the back surface using hand-fastened screws to enable easy installation and removal.

In one embodiment of the invention, the connecting hooks are designed with a curved or tapered profile to facilitate smooth insertion and self-aligning engagement with the predefined slot in the stand bracket.

In one embodiment of the invention, the stand bracket is equipped with locking grooves or recesses that precisely accommodate the connecting hooks of the modular connecting board, ensuring a secure fit.

In one embodiment of the invention, the stand assembly incorporates a locking hinge mechanism that allows the panel to be tilted or rotated for optimal light exposure angles.

In one embodiment of the invention, the stand assembly includes a height-adjustable mechanism that allows the modular light therapy panel to be positioned at varying elevations for optimal therapy coverage.

In one embodiment of the invention, the modular connecting board is configured to provide a distance between the modular light therapy panel and the stand assembly.

In one embodiment of the invention, the modular connecting board comprises a plurality of ventilation holes for dissipating heat generated by the light source.

In the context of this specification, the term ‘modular light therapy panel’ refers to any therapeutic illumination device designed to emit light at specific wavelengths for medical, cosmetic, or wellness applications. The modular nature of the panel allows for interconnection with additional panels or accessories to expand or customize treatment coverage. The term ‘light source’ refers to any component emitting therapeutic wavelengths, including light-emitting diodes (LEDs), organic LEDs (OLEDs), laser diodes, infrared emitters, and full-spectrum lamps.

In the context of the specification, the term “LED” refers to one or more light-emitting diode (LED) elements that are electrically connected and configured to emit light of specific wavelengths (including visible to IR wavelengths) suitable for therapeutic purposes. The LED element or module may include drive circuitry, heat dissipation structures, and optical elements such as lenses or diffusers to control light distribution.

In the context of this specification, terms like “light,” “radiation,” “irradiation,” “emission” and “illumination,” etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorised into several categories according to respective wavelength ranges, which are again envisaged to be within the scope of this invention. A commonly used subdivision scheme for IR radiation includes Near IR (750-1400 nm), Short-Wavelength IR (1400-3000 nm), Mid-Wavelength IR (3000-8000 nm), Long-Wavelength IR (8000-15,000 nm), and Far IR (15,000-1,000,000 nm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source,” etc., refers to a source emitting coherent laser light, or light-emitting diodes (“LEDs”). The term “light therapy” refers to light generated from any of the sources, such as lasers, LED sources, or Super luminous diodes (“SLD”), or Organic light-emitting diodes (OLED).

In the context of the specification, the term “lamp board” refers to a printed circuit board or similar substrate on which one or more light sources are mounted and electrically connected to a power source, such as a rechargeable battery.

In the context of the specification, the term “light source” refers to any active component, such as a light-emitting diode (LED), capable of emitting phototherapy light (e.g., red, infrared, or blue) toward the scalp.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others. The LEDs may also include UV-emitting diodes specifically selected for antimicrobial action in disinfection modules integrated into the multifunctional physiotherapy comb device.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminium, Boron, Zinc Selenide, etc., in pure form or doped with elements such as Aluminium and Indium, etc. For example, red and amber colours are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider colour gamut. White and other colored lightings may also be produced using phosphor coatings such as Yttrium Aluminium Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557B 2 , titled “Roll-to-roll fabricated light sheet and encapsulated semiconductor circuit devices”, which is included herein in its entirety by reference.

In the context of this specification, the term ‘stand assembly’ refers to any structural framework designed to support and position one or more modular light therapy panels. The stand assembly may include caster wheels, wall-mounted brackets, ceiling suspension systems, tabletop mounts, or foldable tripod stands. It may be constructed from metal, reinforced plastic, composite materials, or hybrid structures for durability and stability, with bases incorporating fixed legs, suction pads, weighted bases, or anti-slip rubber feet for enhanced support.

In the context of this specification, the term ‘stand bracket’ refers to any component that serves as an attachment point for the modular light therapy panel. Stand brackets may feature mounting slits, adjustable clamps, sliding rails, magnetic couplings, or pivoting joints for flexible positioning. Additional elements may include shock-absorbing components, rubberized coatings, or vibration-dampening materials to minimize mechanical stress.

In the context of this specification, the term ‘fastening mechanism’ refers to any system that secures modular components without requiring additional tools. Fastening mechanisms may include rotating knobs, lever locks, spring-loaded clamps, quick-release buttons, or threaded locking pins, made from plastic, stainless steel, rubber-coated materials, or composite alloys for enhanced grip and longevity.

In the context of this specification, the term ‘height adjustment mechanism’ refers to any system that allows users to modify the vertical positioning of the modular light therapy panel. This may include pre-drilled screw holes, telescopic columns, ratcheting arms, gas-spring height adjusters, or motorized height controls. Similarly, the term ‘tilt-adjustment mechanism’ refers to any feature that enables the angling of the light therapy panel, including hinged brackets, ball-and-socket joints, friction-based pivots, or electronically controlled tilt actuators.

In the context of this specification, the term ‘mobility mechanism’ refers to any system that enables the movement and repositioning of the stand assembly. While the disclosed embodiment includes caster wheels, alternative implementations may involve gliding tracks, retractable wheels, skid plates, or hoverboard-style movement systems. These mechanisms may feature shock absorbers, noise-dampening rubber coatings, or multidirectional rotation for improved maneuverability.

In the context of this specification, the term ‘hanging mechanism’ refers to any system that enables modular light therapy panels to be suspended from a fixed structure such as a wall, ceiling, or door mount. Hanging mechanisms may include adjustable tension cables, retractable pulley systems, adhesive-backed mounting plates, or foldable wall brackets, with features such as anti-slip coatings, reinforced load-bearing elements, or automatic tension adjustments for secure placement.

In the context of this specification, the term ‘stability mechanism’ refers to any feature that prevents unintended movement or misalignment of modular light therapy panels. Stability mechanisms may include guiding rails, alignment pins, magnetic stabilizers, counterweights, dual-locking hinges, or gyroscopic balancing systems, ensuring secure positioning even with frequent adjustments.

In the context of this specification, the term ‘housing’ refers to any enclosure containing the light source, control assembly, and associated circuitry. The housing may have rectangular, cylindrical, hexagonal, flexible, or wrap-around configurations, composed of metal alloys, heat-resistant polymers, reinforced composites, or ceramic-based materials. Additional features may include textured surfaces, reflective coatings, or ergonomic contours to enhance usability.

In the context of this specification, the term ‘control assembly’ refers to any system used to regulate light intensity, duration, frequency, or operational modes. This may include physical buttons, rotary dials, touchscreen interfaces, remote controls, mobile applications, or voice-activated systems, with features such as programmable timers, preset therapy modes, or AI-driven adaptive settings for personalized treatments.

In the context of this specification, the term ‘heat dissipation system’ refers to any mechanism designed to prevent overheating and maintain optimal operating conditions. Cooling solutions may include ventilation holes, heat sinks, thermally conductive materials, fans, liquid cooling channels, phase-change materials, or thermoelectric cooling modules.

In the context of this specification, the term ‘power switch’ refers to any means of activating or deactivating the device, including toggle switches, capacitive touch sensors, remote controls, or software-based activation. Similarly, the term ‘electrical connection’ includes wired connections (AC/DC, USB-C, proprietary interfaces) or wireless power solutions (inductive charging, battery-based operation).

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1A illustrates a front view of a modular light therapy panel, in accordance with an embodiment of the present invention.

FIG. 1B illustrates a back view of the modular light therapy panel, in accordance with an embodiment of the present invention.

FIG. 1C illustrates a perspective view of the modular light therapy panel, in accordance with an embodiment of the present invention.

FIG. 2A shows a connection of a connecting bracket at the back of the modular light therapy panel, in accordance with an embodiment of the present invention.

FIG. 2B shows an exploded view of the connection between the connecting bracket at the back side of the modular light therapy panel, in accordance with an embodiment of the present invention.

FIG. 3 illustrates a plurality of modular light therapy panels connected horizontally through one or more connecting brackets, in accordance with an embodiment of the present invention.

FIG. 4 illustrates a plurality of modular light therapy panels connected vertically through the connecting bracket, in accordance with an embodiment of the present invention.

FIG. 5A illustrates a modular connecting board connected to the back side of the modular light therapy panel, in accordance with an embodiment of the present invention.

FIG. 5B is an exploded view illustrating the connection between the modular connecting board and the modular light therapy panel, in accordance with an embodiment of the present invention.

FIGS. 6A and 6B illustrate a stand for mounting one or more modular light therapy panels, in accordance with an embodiment of the present invention.

FIG. 7 illustrates the modular light therapy panels mounted on the stand, in accordance with an embodiment of the present invention.

FIG. 8 illustrates a plurality of horizontally connected modular light therapy panels mounted on the stand, in accordance with an embodiment of the present invention.

FIG. 9 illustrates a plurality of vertically connected modular light therapy panels mounted on the stand, in accordance with an embodiment of the present invention.

FIG. 10 shows a modular light therapy panel with a mounting bracket illustrating a hanging mechanism, in accordance with an embodiment of the present invention.

FIG. 11 shows the structure of a bracket body connecting two mounting members, in accordance with an embodiment of the present invention.

FIG. 12 shows an exploded view of FIG. 4, in accordance with an embodiment of the present invention.

FIG. 13 shows a clamping member of the bracket body in accordance with an embodiment of the present invention.

FIG. 14 shows a plurality of modular light therapy panels connected to the hanging mechanism in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide a modular light therapy panel and a system, designed to provide customizable and expandable therapeutic illumination solutions. The modular light therapy panel comprises an array of light-emitting diodes (LEDs) arranged on a front surface, enclosed within a housing containing a light source and a control assembly. The back surface of the panel features an interlocking mounting slot with a curved, wave-like contour and multiple circular cutouts, facilitating secure attachment to a stand assembly or additional panels. The back surface further includes pre-drilled holes for mounting a modular connecting board, which enables tool-free installation via rotating screw knobs and aligns with a stand assembly mounting base featuring grooved channels for precise positioning.

The disclosed modular lighting panel system enables the interconnection of multiple light therapy panels using a bracket secured by knobs inserted into mounting holes within the interlocking slots. The bracket, made from reinforced materials, enhances structural support and allows for both horizontal and vertical expansion. Integrated metal bushings and threaded inserts improve durability and resistance to wear from repeated attachment cycles. The interlocking slots feature beveled edges for seamless engagement, while the knobs incorporate textured gripping surfaces and spring-loaded locking mechanisms to prevent unintended loosening due to vibration.

The stand assembly includes a height-adjustable mechanism for optimized therapy coverage and incorporates a stand bracket with predefined slots designed to engage with connecting hooks on the modular connecting board. The connecting hooks utilize a spring-loaded or latch-based locking mechanism to ensure secure attachment while allowing easy removal. Additionally, the stand assembly features a locking hinge mechanism for precise tilt and rotation adjustments, enabling customizable light exposure angles.

This modular system provides an adaptable, tool-free configuration that allows users to expand and adjust their light therapy setup according to their needs, ensuring optimal coverage and usability across various therapeutic applications.

In an embodiment of the present invention, a modular Light Therapy Panel system that allows users to configure and arrange multiple panels in various configurations, such as vertical or horizontal, for optimized light therapy applications is provided. The system incorporates innovative connection mechanisms that enable users to attach, detach, and customize panel placements according to personal preferences, space availability, and therapy requirements. This vertical and horizontal alignment of the panel makes it easier to cover the large treatment area.

The modular nature of the light therapy panels enables users to integrate them with stands, wall mounts, or other supporting structures that allow for height adjustment. This flexibility ensures that the therapy panels can accommodate users of different heights and treatment needs, enhancing the overall efficiency and convenience of the system. Furthermore, the stand features rotatable components, enabling easy relocation and enhancing portability. This design improves mobility and user convenience, as the panel can be effortlessly attached or detached as needed.

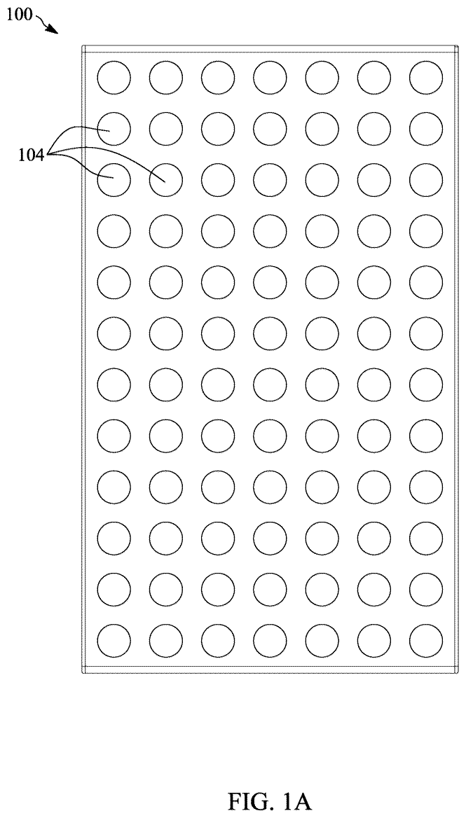

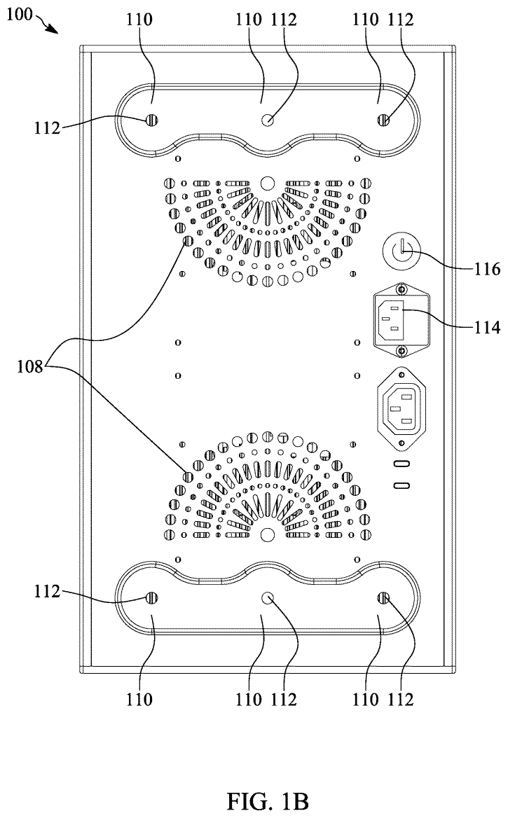

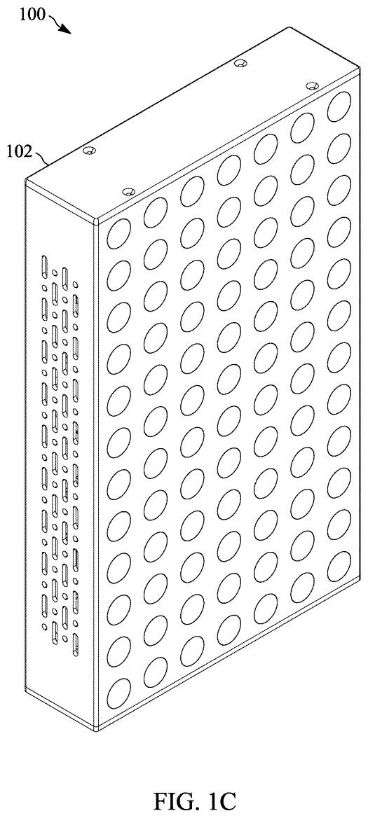

Referring to FIG. 1A, FIG. 1B and FIG. 1C, the modular light therapy panel 100 integrates a housing 102 having a light source 104 and a control assembly to control the light source 104. The front surface of the modular light therapy panel comprises an array of light-emitting diodes arranged in a structured manner. The light source 104 is configured to emit therapeutic wavelengths of light, optimizing the effectiveness of the treatment through uniform light distribution. The modular light therapy panel 100 has a rectangular housing 102 with curved or sharp edges and is designed with high-quality, heat-dissipating material to prevent overheating during extended use. The light source 104 includes a plurality of light-emitting diodes (LEDs) arranged in a matrix configuration on the front surface of the panel. The plurality of LEDs extends substantially to the peripheral edges of the panel so that, when two or more modular light therapy panels 100 are connected or joined together, the distance between adjacent LEDs on adjoining panels is minimized. This configuration ensures a uniform and continuous light output across the entire treatment surface, enhancing the therapeutic effect.

The back portion of the modular light therapy panel 100 includes a plurality of ventilation holes 108 to dissipate heat during the treatment. The back surface of the modular light therapy panel 100 includes the interlocking mounting slots 110 at both the upper and the lower ends. The back side also comprises a plurality of screw holes 112 that facilitate easy attachment of connecting elements. Additionally, the backside of the modular light therapy panel 100 is integrated with the wire input slot 114 for electrical connection. The back surface is configured in a way that it can attach to multiple attachment elements, such as straps, a stand assembly, or a second light therapy panel.

The interlocking mounting slot 110 is designed with a curved, wave-like contour incorporating two or more circular cut-outs, enabling seamless attachment to other modular light therapy panels 100 or mounting structures. This design allows users to adjust and secure the connecting bracket 118 at various positions based on their specific needs. The curved or scalloped shape ensures that the connecting bracket 118 rests securely in place before tightening, preventing misalignment and enhancing overall stability. By integrating multiple circular cut-outs, the interlocking mounting slot 110 accommodates different mounting configurations. The interlocking mounting slot 110 is reinforced with a beveled or chamfered edge to facilitate smooth engagement and disengagement of the connecting bracket 118, making it adaptable for various applications while ensuring a firm and reliable connection.

The modular light therapy panel 100 is enclosed in a durable, lightweight rectangular frame that enhances portability while maintaining structural integrity. The sides of the modular light therapy panel 100 are also integrated with ventilation holes 108 to prevent overheating of the modular light therapy panel 100. A display 120 is placed above the ventilation holes 108, which allows the user to customize the duration and the intensity of the light therapy. The modular light therapy panel 100 is designed to be modular, allowing users to combine multiple units to create customized configurations based on therapy requirements. A power switch 116 to turn the device ON/OFF is provided on the back or side wall of the modular light therapy panel 100.

By allowing heat to escape, these perforations help maintain optimal internal temperatures, preventing potential damage to electronic components and ensuring sustained performance. The ventilation holes 108 are strategically positioned around high-heat-generating components and contribute to energy efficiency by reducing the need for active cooling mechanisms, thereby minimizing power consumption. Additionally, they enhance the panel's durability by preventing thermal stress on internal circuitry, ensuring long-term reliability. The placement and size of the ventilation holes 108 are designed to optimize heat dissipation while maintaining the structural integrity and aesthetic appeal of the panel.

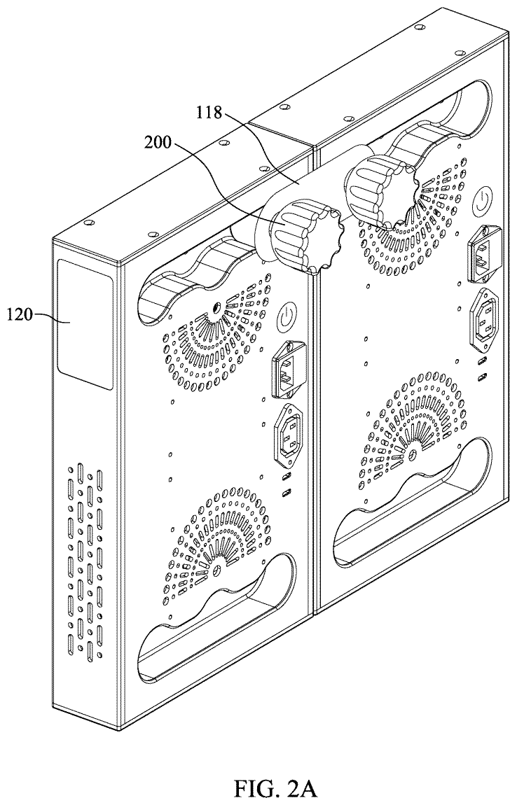

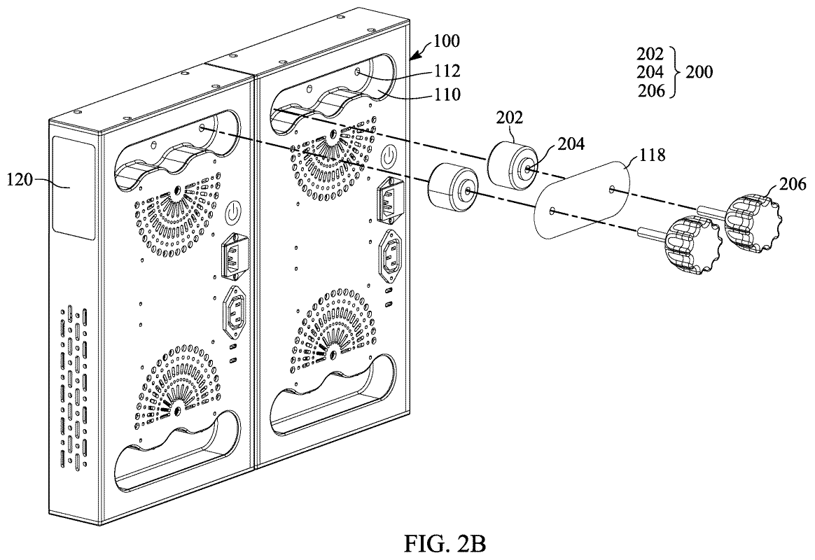

Referring to FIG. 2A, the image illustrates the connection of a connecting bracket 118 at the back of the modular light therapy panel 100. The connecting bracket 118 is designed to secure two or more modular light therapy panels 100 together in a stable configuration. It is affixed to the back of the panel through a series of mounting members 200, which fit into the screw holes 112 in the interlocking mounting slot 110. The connecting bracket 118 is a plate-like structure with semi-circular edges and is integrated with two screw holes 112 at each of the edges.

Additionally, the interlocking mounting slot 110 houses multiple screw holes 112 and is capable of receiving more than one mounting member 200 at once. The mounting member 200 connects the connecting bracket 118 to the interlocking mounting slot 110, which helps in joining two or more modular light therapy panels 100 together.

The connecting bracket 118 is designed to be placed into the interlocking mounting slot 110 and adjusted along the curved, wave-like contour before being securely fastened with a mounting member 200. This user-friendly design simplifies the installation process, allowing for easy positioning and alignment. The three circular cut-outs enable the threaded shaft of knob 206 to pass through and lock into place, ensuring a stable and secure connection. Additionally, this design prevents slippage or misalignment after installation, maintaining the intended position of the connecting bracket 118. The scalloped or wave-like shape further enhances versatility by accommodating different connecting brackets 118 widths or positions, making it an adaptable and reliable mounting system for various applications.

Referring to FIG. 2B, an exploded view of the connection between the connecting bracket 118 at the back side of the modular light therapy panel 100 is shown. The screw holes of both the interlocking mounting slot 110 and the connecting bracket 118 are aligned together and fixed through a mounting member 200. The mounting member comprises a mounting base 202 having a through-hole 204, and a knob 206 with a shaft. The shaft of the knobs 206 is passed through the screw holes 112 present in the connecting place, and the base is positioned below the connecting bracket 118. The shaft of the knob 206, after passing through the hole in the connecting bracket 118, passes through the through-hole 204. The mounting base 202 is secured in the interlocking mounting slot 110, and the shaft of the knob 206 is fixed in the screw holes 112 present in the interlocking mounting slot 110 by rotating it.

The mounting process begins with the placement of the mounting base 202 into the designated interlocking mounting slot 110, aligning it with one of the circular cut-outs. The connecting bracket 118 is positioned over the mounting base, such that the hole in the connecting bracket 118 aligns with the through-hole 204 in the mounting base 202. Once positioned, the knob's 206 shaft is inserted through the selected cut-out and screwed into the connecting bracket's 118 mounting hole. The shaft may be threaded or non-threaded. In the case of a non-threaded shaft, the diameter of the hole of the shaft to pass the knob screw is more than the diameter of the screw of the knob. As the knob 206 is tightened, the wave-like edges of the slot help prevent movement, creating a stable, vibration-resistant connection. This design ensures a secure fit while allowing for easy adjustments when needed.

The knob 206 features a knurled or textured gripping surface, a star or scallop-shaped outer surface, providing an ergonomic grip for easy manual tightening and loosening. This design ensures tool-free operation, allowing users to adjust the mounting system with minimal effort. The knobs 206 likely incorporate a shaft extending from the bottom, either as an integrated bolt (male thread) or a threaded insert (female thread), which passes through the connecting bracket 118 and into a pre-threaded hole or captive nut in the modular light therapy panel 100. As the knob 206 rotates clockwise, the threads engage, pulling the connecting bracket 118 and the mounting base 202 in the modular light therapy panel 100 tightly together to create a firm and secure connection without requiring additional tools. This design allows for easy installation and adjustments, making it ideal for modular systems where frequent repositioning or removal is necessary, such as light therapy panels, monitor stands, or mounting rigs. To loosen or adjust the connecting bracket 118, the user simply turns the knob 206 counter-clockwise, releasing the tension and enabling quick disassembly or repositioning. Some knobs 206 may incorporate a spring-loaded or captive washer to evenly distribute pressure and prevent overtightening, ensuring durability and ease of use. Depending on the system's specifications, the threading may follow metric standards (e.g., M6, M8) or imperial measurements (e.g., ¼″-20), ensuring compatibility with various mounting configurations.

The material of the knob 206 varies based on its intended application and durability requirements, such as plastic knobs, typically made from ABS, nylon, or polypropylene, which are lightweight, corrosion-resistant, and suitable for non-load-bearing applications where ease of use and cost efficiency are priorities but for more demanding applications requiring higher torque and durability, metal knobs made from aluminum, stainless steel, or zinc alloy can be preferred due to their strength and resistance to wear. Additionally, rubber-coated knobs offer enhanced grip and comfort, making them ideal for ergonomic applications where frequent manual adjustments are needed. The choice of material ensures that knob 206 meets functional and environmental requirements while providing an optimal user experience.

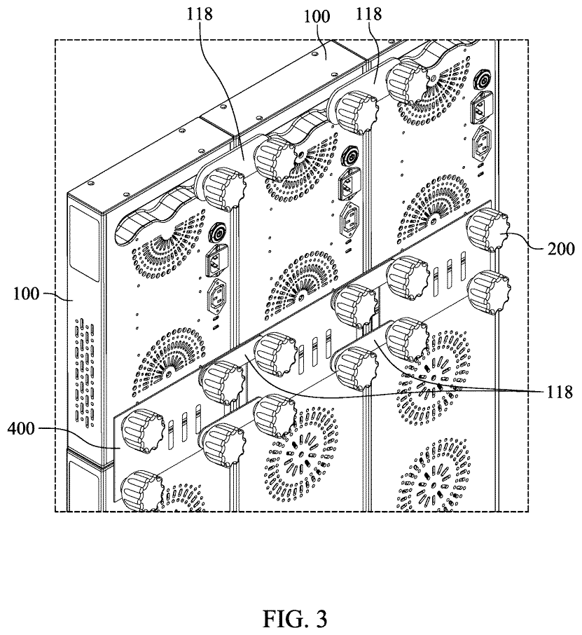

FIG. 3 illustrates a plurality of modular light therapy panels 100 connected horizontally through one or more connecting brackets 118. In this configuration, the interlocking mounting slot 110 of two or more adjacent modular light therapy panels 100 are aligned together, and a connecting bracket 118 is horizontally secured over the screw holes 112. Once the screw holes 112 of both the interlocking mounting slot 110 and connecting bracket 118 are properly positioned over each other, the mounting member 200 is inserted and fixed to secure the placement, ensuring a strong attachment.

The horizontal configuration ensures uniform light distribution across the connected modular light therapy panels 100, enhancing therapeutic effectiveness by covering a broader surface. Additionally, the design allows users to customize the number of connected modular light therapy panels 100 based on their specific therapy needs, whether for localized or full-body treatments. The modular nature of the system enables users to expand their setup easily by attaching additional panels without requiring complex tools or permanent modifications.

The connecting brackets 118 provide a firm and stable connection between the modular light therapy panels 100, ensuring that they remain securely aligned even during prolonged use. The system's scalability makes it suitable for both personal and professional applications, such as clinical settings where larger therapy panels are needed.

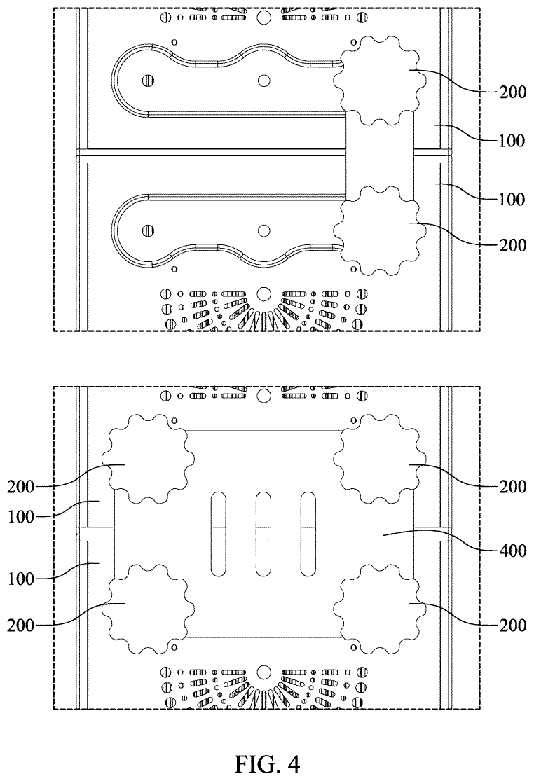

FIG. 4 illustrates a plurality of modular light therapy panels 100 connected vertically through the long connecting plate 400. In this configuration, the modular light therapy panels 100 are securely fastened using mounting members 200 that fit into screw holes 112 in the interlocking mounting slot 110 by stacking multiple modular light therapy panels 100. In this arrangement, the long connecting plate 400 is arranged in a vertical orientation over the interlocking mounting slot 110. Once the screw holes 112 are properly aligned, both elements are fixed together using the mounting member 200. By stacking up multiple modular light therapy panels 100, the user can achieve an extended treatment area without occupying excessive horizontal space, making it ideal for home use, clinics, or compact therapy environments. The vertical stacking mechanism ensures a stable and secure connection between the modular light therapy panels 100, preventing misalignment or structural instability. The long connecting plate 400 used in this configuration is designed to evenly distribute the weight of the attached panels, ensuring that they remain securely fastened.

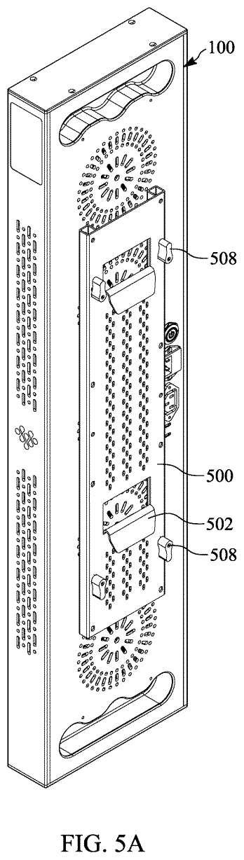

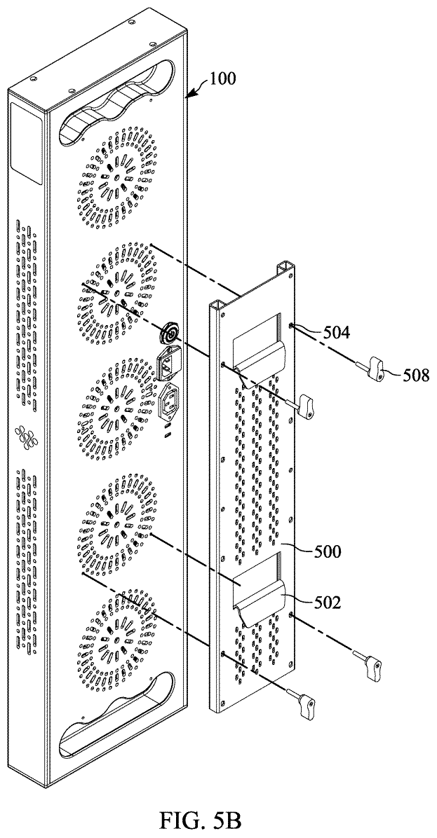

Both the horizontal and vertical arrangements are possible due to the positions of the interlocking mounting slot 110, as they are arranged at both the upper and the lower portions of the modular light therapy panel 100. The interlocking mounting slot 110 has a hollow space engraved with more than one screw hole 112. The interlocking mounting slot 110 can also be used to hold a modular light therapy panel 100 while relocating or positioning for a treatment. FIG. 5A illustrates a modular connecting board 500 connected to the back side of the modular light therapy panel 100. FIG. 5B is an exploded view that illustrates the connection between the modular connecting board 500 and the modular light therapy panel 100. The modular connecting board 500 has a long rectangular shape and has two cut-outs. These cut-outs accommodate connecting clamping hooks 502. The connecting clamping hooks 502 are utilized in hanging the modular light therapy panels 100 to the stand, ensuring a secure interlocking mechanism.

The modular connecting board 500 features a series of evenly spaced mounting holes 504 that fit over the pre-drilled holes on the modular light therapy panel 100 for secure mounting and flexibility. The mounting holes 504 enable screws/fasteners 508 to firmly attach the modular connecting board 500 to the modular light therapy panel 100 or stand bracket 510, ensuring stability. The mounting holes 504 can be elongated in shape to adjust the modular connecting board 500 while mounting on the modular light therapy panel 100. Additionally, the mounting slits 506 accommodate clamping hooks 502, creating a secure interlocking mechanism that enhances structural integrity. The stand bracket 510 and adjustable mounting slits 506 provide further versatility, allowing users to position the panels at different heights or angles based on specific requirements. This design ensures both ease of installation and adaptability for various mounting configurations.

The modular connecting board 500 is also responsible for one of the vertical arrangements of one or more modular light therapy panels 100. This arrangement requires the modular light therapy panels 100 to be stacked on one another. These stacked panels are fixed through modular connecting boards 500 and screws/fasteners 508. It is fixed in a way that bridges the lower portion of the first panel and the upper portion of the second panel, ensuring a stable vertical connection.

The modular light therapy panel 100 and the modular connecting board 500 are both designed with a series of evenly spaced mounting holes 504, ensuring secure attachment to the modular light therapy panel 100 or stand bracket 510. These mounting holes 504 allow screws/fasteners 508 to be used for firm positioning, while the cut-outs accommodate clamping hooks 502, providing a stable interlocking mechanism. The modular connecting board 500 comprises a reinforced metallic or polymer structure to provide enhanced rigidity and load-bearing capacity when mounted on the stand 600.

In addition to mounting holes 504, the modular connecting board 500 incorporates designated cut-outs specifically designed for clamping hooks 502. These clamping hooks 502 slide into the mounting slits 506 that are pre-designed on the stand bracket 510, enabling a firm and reliable hold without the need for excessive tightening. This mechanism ensures a secure fit while also allowing for quick assembly and disassembly, making it convenient for users who frequently adjust or rearrange their setup.

To further enhance stability, the modular connecting board 500 utilizes mounting members 200 and screws/fasteners 508. The rotating mounting member 200 is designed for tool-free operation, allowing users to secure the modular connecting board 500 with ease. These mounting members 200 provide a quick and reliable locking mechanism, while additional screw/fasteners 508 reinforce the overall structural integrity. This combination ensures a firm, immovable, and secure connection while maintaining the flexibility needed for modular setups. The modular connecting board 500 additionally includes integrated cable management clips or channels to organize and secure power and control wiring.

The modular connecting board 500 is specifically engineered for seamless integration with stand brackets 510. These stand brackets 510 feature elongated mounting slits 506 that align with the connecting clamping hooks 502 on the board, ensuring a precise and stable attachment. The design allows for both horizontal and vertical mounting, enabling scalable configurations to accommodate different panel arrangements.

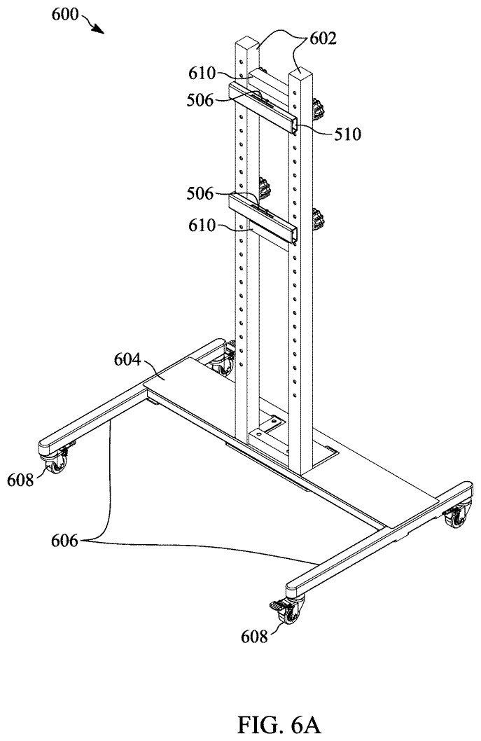

Accordingly, FIGS. 6A and 6B illustrate a stand 600 for mounting one or more modular light therapy panels. The stand 600 is designed to provide a stable and adaptable support structure for modular light therapy panels 100. It consists of multiple components that work together to ensure secure attachment, easy adjustments, and enhanced usability. The stand integrates two vertical stand-bars 602, stand brackets 510, and a base platform 604. The base platform 604 consists of two arms 606 with caster wheels 608. The stand-bars 602 have two joining rods 610, connecting both the vertical stand-bars 602. The vertical stand-bars 602 and the base platform 604 are fixed through anchors 612. The stand 600 consists of multiple stand brackets 510. Further, the stand bracket 510 serves as the primary structural element, offering predefined mounting slits 506 that accommodate the connecting clamping hooks 502 from the modular connecting board 500, and is detachable and includes a quick-release mechanism for rapid reconfiguration of modular light therapy panels. This stand bracket 510 is engineered to support multiple modular light therapy panels 100, allowing for both horizontal and vertical configurations based on user preferences and therapy needs. The stand bracket 510 may incorporate integrated dampening elements to reduce mechanical stress and vibrations between interconnected panels. The stand 600 has a plurality of screw holes 112 which connect the modular light therapy panels 100 and the stand 600 together with the help of the mounting member 200. These multiple screw holes 112 allow the user to adjust the height of the modular light therapy panel 100.

At the core of the system is the modular connecting board 500, which attaches to the stand bracket 510 using mounting member 200 and screw/fasteners 508. The perforated design of the modular connecting board 500 improves ventilation, preventing overheating while also reducing the overall weight of the structure. These features make it an ideal component for modular systems that require frequent adjustments and reconfigurations.

To enhance ease of installation, mounting members 200 are used for tool-free attachment of the modular connecting board 500 to the stand bracket 510. These mounting members 200 allow users to quickly secure or adjust the modular connecting board 500 without requiring additional tools. For extra stability, additional mounting members 200 and screws/fasteners 508 are incorporated to prevent unintended loosening due to vibration, ensuring that the panels remain securely in place even when the stand is repositioned.

The base platform 604 and the support structure of the stand 600 play a crucial role in its functionality. The base platform 604 is equipped with caster wheels 608, allowing for easy mobility and repositioning, making it convenient for users to move the setup between different locations. Additionally, the stand 600 features an adjustable height mechanism, enabling users to customize the panel's 100 positions based on therapy requirements. This design can also incorporate a tilt-adjustable mechanism, allowing users to optimize the light therapy angle for maximum effectiveness. These features collectively enhance the usability of the stand assembly, making it a versatile and adaptable solution for light therapy applications.

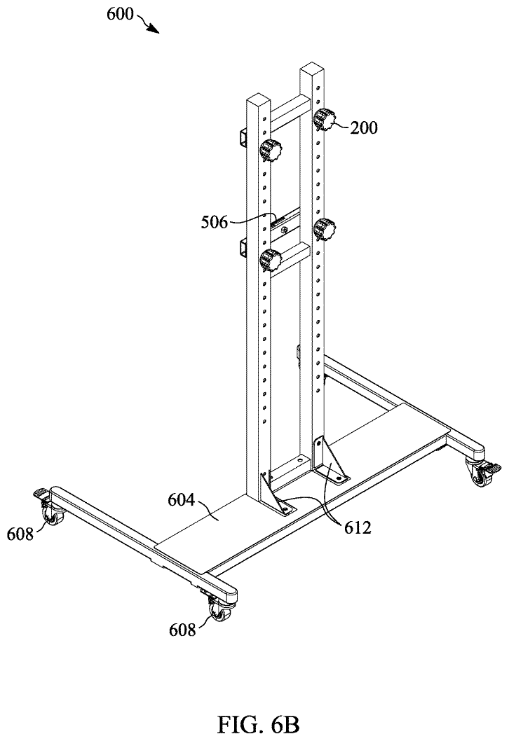

According to FIG. 7, the image depicts the modular light therapy panels 100 mounted on the stand 600. The modular light therapy panel 100 is mounted on the stand 600 with the help of a modular connecting board and stand bracket. The modular connecting board 500 and the stand bracket 510 play a major role in connecting a modular light therapy panel 100 and the stand 600. The modular connecting board 500 has cut-outs, and each cut-out is integrated with a clamping hook 502. The stand bracket 510 is detachable from the vertical stand bar 602 and can be placed according to the user's preferred height.

The assembly and installation process of the stand 600 follows a systematic approach to ensure a stable and secure setup. It involves positioning the stand bracket 510, which is attached to the main vertical support bars 602 of the stand 600 with the help of the rotating mounting member 200. The mounting slits 506 of the stand bracket 510 are aligned with the clamping hooks 502 of the modular connecting board 500 to ensure a seamless connection. Once in place, the mounting member 200 is tightened to lock the modular connecting board 500 securely, and additional screws can be used for extra stability. Furthermore, the stand 600 may include a tilt-adjustment feature that allows the modular light therapy panel 100 to be angled for targeted light exposure.

The modular light therapy panel 100 can be fixed according to the user's height preference using these multiple screw holes 112, as the stand bracket 510 can be separated and placed in different positions. This allows users to raise or lower the modular light therapy panels based on their treatment preferences. This is particularly useful in professional therapy settings where different patients may require different panel positions for optimal exposure. The integrated caster wheels 608 are locked in place to prevent unwanted movement, whereas the stand's sturdy frame and wide base provide additional support, preventing wobbling or instability during use. The secure attachment mechanism ensures that the modular light therapy panels 100 remain in place even if they are repositioned frequently. Additionally, the stand bracket 510 includes guiding rails or alignment pins to facilitate precise positioning and alignment of the modular connecting board 500, and to distribute weight evenly across the structure, reducing strain on the modular connecting board 500 and enhancing the durability of the system.

Moreover, the modular design of the stand-mounted modular light therapy panels 100 enables users to expand their therapy setup by attaching additional panels either horizontally or vertically. This adaptability is especially beneficial for users who require full-body light therapy or wish to target larger treatment areas. The ability to modify the panel arrangement without requiring complex tools or extensive reinstallation makes this system highly user-friendly. Whether used in a clinical setting or at home, the modular connection between the panels and the stand ensures an effective, customizable, and convenient light therapy experience.

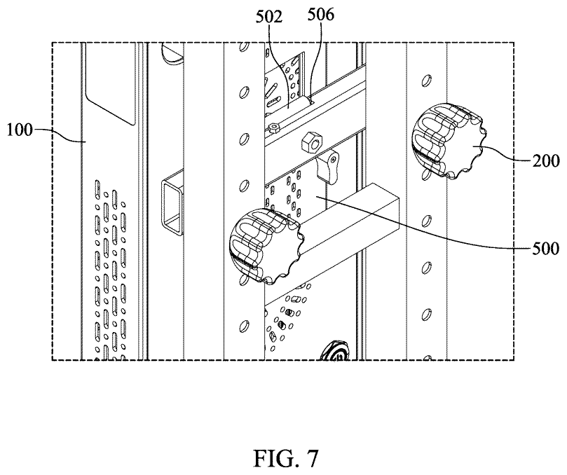

FIG. 8 illustrates a plurality of horizontally connected modular light therapy panels 100 mounted on the stand 600. This configuration requires two or more modular light therapy panels 100 aligned together horizontally or adjacent to each other, and connected on the back through a modular connecting board 500. As mentioned above, similarly, the clamping hooks 502 present on the modular connecting board 500 are inserted into the mounting slits 506 present on the stand bracket 510. This configuration provides a stable framework to maintain alignment and prevent displacement. This arrangement expands the treatment area width by allowing multiple panels to be connected side by side, ensuring uniform light distribution across a larger surface.

The modular design of the modular light therapy panel 100 allows for seamless integration, enabling users to extend the system as needed. Each panel is mounted precisely that accommodate the modular connecting board 500, ensuring a firm and reliable connection. The stand 600 provides additional support by incorporating adjustable height settings, allowing users to customize the positioning of the therapy panels for optimal exposure.

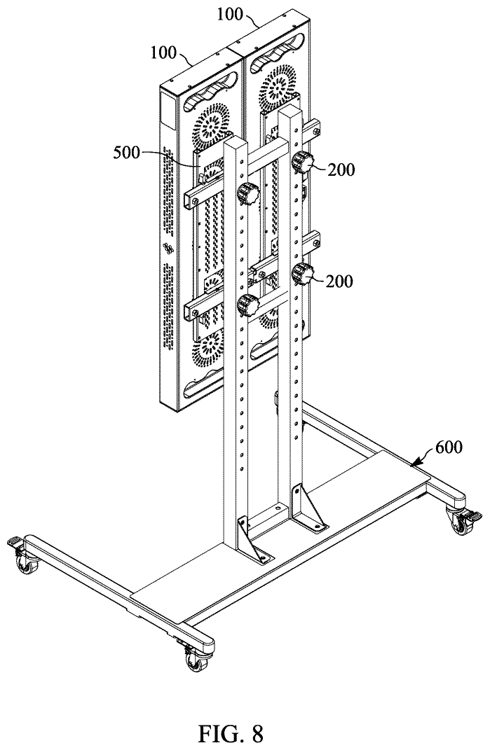

FIG. 9 illustrates a plurality of vertically connected modular light therapy panels 100 mounted on the stand 600. This configuration requires stacking of two or more modular light therapy panels 100, and then the stacked modular light therapy panels 100 are connected via a modular connecting board 500. The hooks/clamping hooks 502 present on the modular connecting board 500 are inserted into the stand bracket 510, which is attached to the stand 600 with the help of the mounting member 200. By stacking multiple modular light therapy panels 100 on top of one another, users can achieve consistent light exposure from head to toe, making it particularly beneficial for applications such as skin treatments, muscle recovery, and circadian rhythm regulation. The vertical arrangement ensures that light is evenly distributed across the entire body length, eliminating the need for repositioning or multiple therapy sessions to cover different areas.

The vertical connection of the modular light therapy panel 100 is achieved using the modular connecting board 500 and secure fastening mechanisms. These components provide structural stability, ensuring that the stacked modular light therapy panels 100 remain firmly in place during operation. This adaptability makes the system ideal for both seated and standing treatments, catering to different user preferences and clinical applications.

Unlike horizontally arranged modular light therapy panels 100 that require additional width, the vertical setup maximizes the use of vertical space, making it suitable for compact environments such as home therapy setups, clinics, and wellness centers. The secure mounting system prevents wobbling or misalignment, ensuring a stable and uninterrupted therapy session. Additionally, the modular nature of the system allows for easy expansion, enabling users to add or remove modular light therapy panels 100 as needed based on their treatment goals.

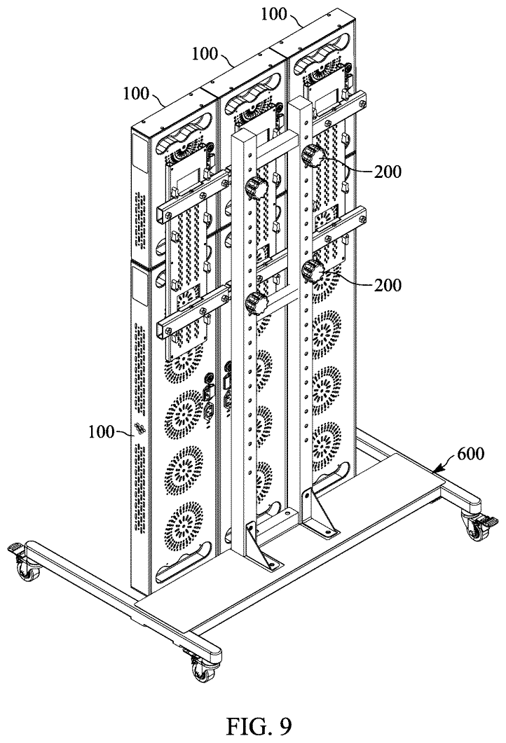

In an embodiment of the present invention, a hanging mechanism for fixing the modular light therapy panel on a support is provided. Referring to FIG. 10, the hanging mechanism comprises a mounting bracket connected to the modular light therapy panel. The mounting bracket facilitates the installation of the modular light therapy panel 100, which emits light for phototherapy. The mounting bracket includes at least two bracket bodies 300, positioned on both sides of the modular light therapy panel 100.

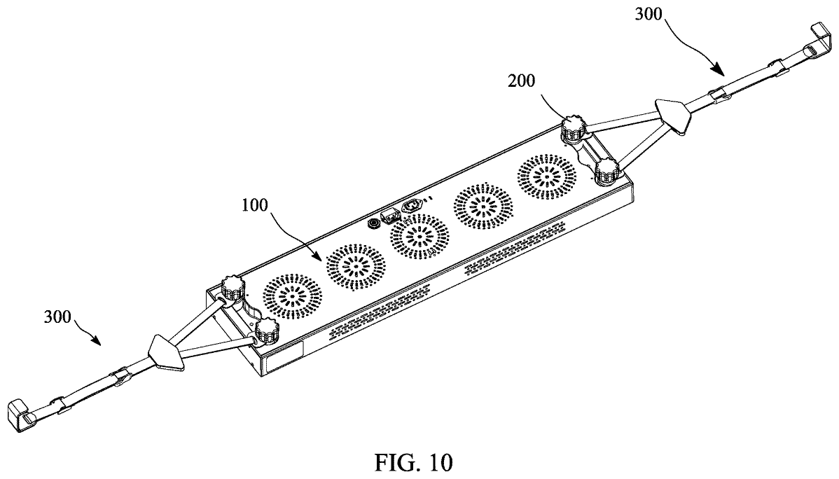

Referring to FIG. 11, each bracket body 300 comprises a connector 302, a mounting plate 324, and a hook 330. One end of the connector 302 is connected to the mounting plate 324, and the other end is connected to the hook 330. The mounting plate 324 is configured to attach to the modular light therapy panel 100, while the two hooks 330 are mountable onto a desired surface (for example, a door panel or a mounting plate). During installation, the two mounting plates 324 are attached to the ends of the modular light therapy panel 100 and suspended from the surface via the two hooks 330. This arrangement enables convenient mounting and removal of the modular light therapy panel 100. Compared with traditional fasteners such as screws, the use of the mounting bracket simplifies assembly and disassembly.

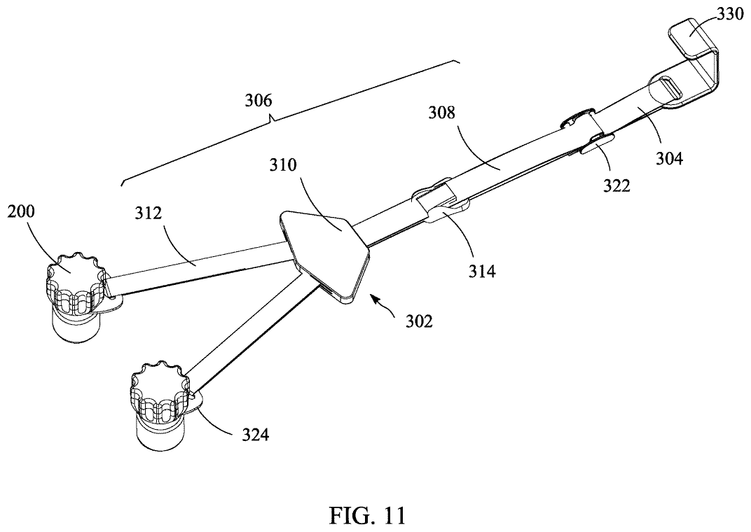

Referring to FIGS. 11 and 12, each connector 302 includes a first connecting cord 304, a second connecting cord 306, and a length adjustment member 322 connecting the two cords. The end of the first connecting cord 304 opposite the length adjustment member 322 is connected to the hook 330, while the end of the second connecting cord 306 opposite the length adjustment member 322 is connected to the mounting plate 324. This configuration allows the overall length of the connector 302 to be adjusted via the length adjustment member 322, thereby accommodating different installation requirements and modular light therapy panels 100 of various sizes. This enhances the adaptability and usability of the mounting bracket.

Referring to FIGS. 10 and 11, each second connecting cord 306 comprises a first cord body 308, a connecting base 310, and at least two second cord bodies 312. One end of each first cord body 308 is connected to the corresponding length adjustment member 322, and the other end is connected to one side of the connecting base 310. Each second cord body 312 is connected between the opposite side of the connecting base 310 and the mounting plate 324. The number of second cord body 312 corresponds to the number of mounting plates 324.

In this configuration, by providing at least two second cord bodies 312 connected respectively to at least two mounting plates 324, each bracket body 300 is connected to the end of the modular light therapy panel 100 at multiple points, thereby improving overall stability and minimizing vibration during use.

Referring to FIGS. 11 and 12, each second connecting cord 306 further includes a clamping member 314 installed on the first cord body 308. The cooperation of the clamping member 314 and the length adjustment member 322 enables fine-tuning of the cord length and secure fixation. In one embodiment, the length adjustment member 322 is a Japanese-style buckle, through which the first cord body 308 is threaded. Specifically, the first cord body 308 includes a connecting section linked to the connecting base 310 and an adjusting section inserted through the buckle. The clamping member 314, mounted on the connecting section, secures the adjusting section in place.

In alternative embodiments, the length adjustment member 322 may be a long plate with multiple mounting holes along its length, allowing the first cord body 308 to be adjusted by inserting it into different holes. The specific structure of the length adjustment member 322 is not limited.

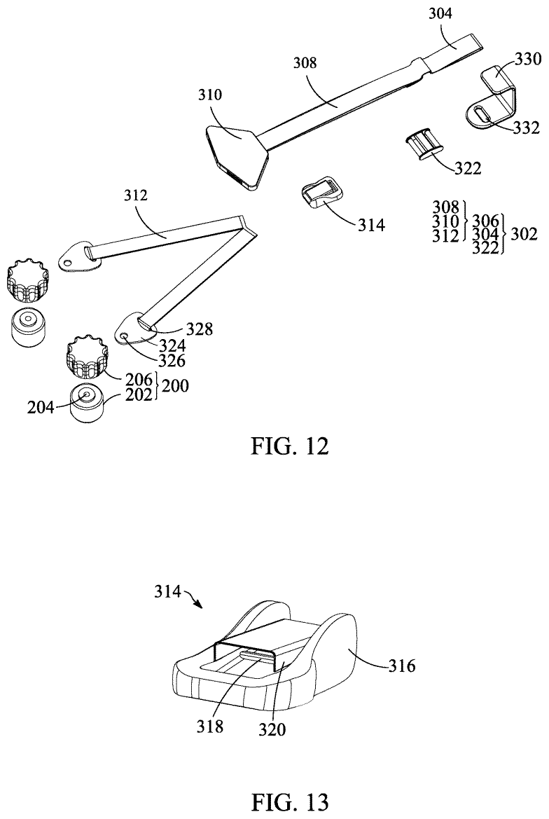

Referring to FIG. 13, each clamping member 314 includes an adjusting base 316, a first clamping seat 318, and a second clamping seat 320. The adjusting base 316 allows one end of the first cord body 308 to pass through. The first clamping seat 318 is fixed on the adjusting base 316, while the second clamping seat 320 is hinged to the adjusting base 316 above the first clamping seat 318. When the second clamping seat 320 rotates away from the first clamping seat 318, the clamping member 314 enters an open state, releasing the first cord body 308 and allowing length adjustment. When rotated back toward the first clamping seat 318, the clamping member 314 enters a closed state, securely holding the cord. Thus, the cord length can be finely adjusted by repositioning the clamping member 314.

In alternative embodiments, the clamping member 314 may be a D-ring connecting the first cord body 308 and the connecting base 310, allowing adjustable locking positions, or a Velcro strap for securing the adjusting section of the cord. The structure of the clamping member 314 can vary based on application requirements. In some embodiments, one or both of the connecting cords (304 and/or 306) are elastic, allowing elastic deformation and tension-based adjustment of the connector 302, thereby enhancing mounting stability.

Referring to FIG. 12, each mounting plate 324 includes a first through-hole 328, which, together with a mounting hole 326, may be located at either end of the plate. The first through-hole 328 allows the second cord body 312 to pass through for installation. Similarly, each hook 330 includes a second through-hole 332 for installation of the first cord body 308. This design facilitates straightforward assembly and secure attachment of the cords.

Referring to FIGS. 10 and 11, the mounting bracket includes two mounting plates 324, each connected to opposite ends of the modular light therapy panel 100. This arrangement, using the two hooks 330 for suspension, enables convenient installation and removal of the modular panel.

Referring to FIG. 11, the modular light therapy panel 100 also includes at least two mounting members 200, each installed at an end of the panel. The two mounting plates 324 are connected respectively to these mounting members 200, providing a detachable connection between the bracket body 300 and the modular light therapy panel 100.

Referring to FIG. 10, two mounting members 200 can be provided at each end of the panel, four in total. Correspondingly, each bracket body 300 is equipped with two mounting plates 324, each connected via a respective second cord body 312. This enhances the overall structural stability of the mounted panel.

Referring to FIGS. 1B and 12, each mounting member 200 includes a mounting base 202 and a knob 206 connected to it. Screw holes 112 are provided at both ends of the modular light therapy panel 100, and through-holes 204 are formed in each mounting base 202. Each knob 206 passes through the corresponding through-hole 204 and locks into the corresponding screw hole 112. Each mounting plate 324 includes the mounting hole 326 through which the knob's 206 shaft passes. The connection between the mounting member 200 and screw hole 112 may be threaded, snap-fit, or other detachable means. The cooperation of the knob 206 and the mounting base 202 ensures secure engagement and firm clamping of the mounting plate 324.

Referring to FIGS. 1B, 2B, and 10, the interlocking mounting slots 110 are provided at each end of the modular light therapy panel 100, each having multiple screw holes 112 at its bottom surface. This design ensures precise positioning of the mounting members 200, improving alignment accuracy and preventing loosening. The mounting base 202 is typically cylindrical, and the interlocking mounting slot 110 has a matching circular cross-section. Alternatively, the mounting base 202 may be a hexagonal or octagonal prism, with corresponding slot shapes. A protective gasket may be provided between the mounting base 202 and the inner wall of the slot to buffer vibration, improve sealing, and prevent wear.

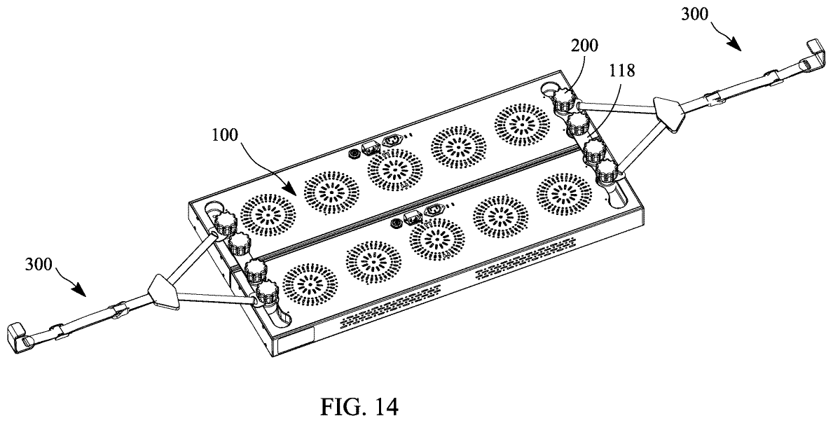

Referring to FIG. 14, the modular light therapy panel 100 may further include at least two connecting brackets 118, each having its ends connected to two adjacent modular light therapy panels 100. Alternatively, two connecting brackets 118 may be provided between adjacent panels. Each connecting plate 400 has openings at both ends through which the corresponding mounting members 200 pass, allowing secure interconnection of panels. This configuration enables multiple modular light therapy panels 100 to be joined together for expanded installation, with one mounting member 200 connecting to the connecting bracket 118 and another connecting to the mounting plate 324.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A modular light therapy panel, comprising:
a housing having a front surface and a back surface;
a light source disposed within the housing;
a control assembly configured to regulate operation of the light source;
at least one interlocking mounting slot formed on the back surface, the interlocking mounting slot having a curved, wave-shaped contour with a plurality of circular cutouts, each of the plurality of circular cutout defining one or more screw hole;
wherein the interlocking mounting slot is configured to receive one or more mounting members for attachment of the modular light therapy panel to a connecting bracket or a mounting bracket.

2. The modular light therapy panel of claim 1, wherein the mounting member comprise a knob and a mounting base with a through-hole; and the connecting bracket or the mounting bracket is secured over the interlocking mounting slot by placing the mounting base in the circular cut-out and allowing a threaded shaft of the knob to pass through a through hole in the connecting bracket/mounting bracket and the mounting base and tightened in the one or more screw holes.

3. The modular light therapy panel of claim 1, wherein the mounting bracket comprises one or more bracket bodies comprising:
a connector having a first end and a second end;
a mounting plate connected to the first end and the configured to engage the interlocking mounting slot through the one or more mounting member;
a hook connected to the second end and configured to attach to a supporting structure.

4. The modular light therapy panel of claim 3, wherein the connector includes a length adjustment member configured to adjust length between the mounting plate and the hook.

5. The modular light therapy panel of claim 1, wherein the interlocking mounting slots facilitate carrying of the modular therapy panel by a user, wherein the user can insert fingers for holding the modular light therapy panel.

6. The modular light therapy panel of claim 1, wherein the connecting bracket is positioned over one of the plurality of circular cut-outs of the modular light therapy panel; and an adjacent modular light therapy panel; and the connecting bracket is configured to connect the modular light therapy panel and the adjacent modular light therapy panel using the one or more mounting members.

7. The modular light therapy panel of claim 1 further comprising a plurality of pre-drilled holes on the back surface for connecting a modular connecting board; and
wherein the modular connecting board includes one or more connecting hooks configured to engage with a predefined slot in a stand bracket of a stand assembly.

8. A modular lighting panel system, comprising:
a plurality of modular light therapy panels, each panel including:
a housing containing a light source on a front surface and a control assembly for controlling the light source;
a back surface configured for attachment to an adjacent modular light therapy panel, the back surface comprising:
at least one interlocking mounting slot positioned at the back surface, extending horizontally across the back surface;
the interlocking mounting slot having a curved, wave-like contour with a plurality of circular cutouts, each cutout including a screw hole for receiving a threaded shaft of a mounting member; and
a connecting bracket positioned over one of the plurality of circular cut-outs of a first modular light therapy panel and a second modular light therapy panel, wherein the connecting bracket is secured by tightening a first mounting member into a first screw hole of the first modular light therapy panel and a second mounting member into a second mounting hole of the second modular light therapy panel.

9. The modular lighting panel system of claim 8, wherein the plurality of circular cutouts includes integrated metal bushings or threaded inserts to enhance durability and resistance to wear during repeated attachment and detachment cycles.

10. The modular lighting panel system of claim 8, wherein the connecting bracket features elongated slots to allow for positional adjustment between the first modular light therapy panel and the second modular light therapy panel.

11. The modular lighting panel system of claim 8, wherein the mounting member includes a spring-loaded locking mechanism to prevent unintended loosening due to vibration or movement during operation.

12. The modular lighting panel system of claim 8, wherein the plurality of modular light therapy panels are configured to allow horizontal and vertical expansion, enables customizable therapy configurations.

13. A modular light therapy panel system, comprising:
a housing having a light source on a front surface and a control assembly to control the light source;
a back surface configured to attach with a stand assembly or a second light therapy panel, wherein the back surface comprises:
at least one interlocking mounting slot on the back surface;
a plurality of pre-drilled holes for connecting a modular connecting board; and
wherein the modular connecting board includes one or more connecting hooks configured to engage with a predefined slot in a stand bracket of a stand assembly.

14. The modular light therapy panel system of claim 13, wherein the modular connecting board is secured to the back surface using hand-fastened screws to enable easy installation and removal.

15. The modular light therapy panel system of claim 13, wherein the connecting hooks are designed with a curved or tapered profile to facilitate smooth insertion and self-aligning engagement with the predefined slot in the stand bracket.

16. The modular light therapy panel system of claim 13, wherein the stand bracket is equipped with locking grooves or recesses that precisely accommodate the connecting hooks of the modular connecting board, ensuring a secure fit.

17. The modular light therapy panel system of claim 13, wherein the stand assembly incorporates a locking hinge mechanism that allows the panel to be tilted or rotated for optimal light exposure angles.

18. The modular light therapy panel system of claim 13, wherein the stand assembly includes a height-adjustable mechanism that allows the modular light therapy panel to be positioned at varying elevations for optimal therapy coverage.

19. The modular light therapy panel system of claim 13, wherein the modular connecting board is configured to provide a distance between the modular light therapy panel and the stand assembly.

20. The modular light therapy panel system of claim 13, wherein the modular connecting board comprises a plurality of ventilation holes for dissipating heat generated by the light source.
